(19) United States
(12) Patent Application Publication
(10) Pub. No.: US 2003/0153493 A1
(43) Pub. Date: Aug. 14, 2003

(54) Combination cancer therapy

(75) Inventor: Qing Fan, Sunnyvale, CA (US)

Correspondence Address: Vinit G. Kathardekar, Pharmacyclics, Inc., 995 E. Arques Avenue, Sunnyvale, CA, 94085, US

(73) Assignee: Pharmacyclics, Inc., Sunnyvale, CA (US)

(21) Appl. No.: 10/319,001

(22) Filed: Dec. 13, 2002

Related U.S. Application Data
(60) Provisional application No. 60/339,649, filed on Dec. 13, 2001.

(51) Int. Cl. A61K 38/14; A61K 31/704; A61K 31/655; A61K 31/555; A61K 31/337

(52) U.S. Cl. 514/8; 514/34; 514/185; 514/449; 514/150; 514/492

(57) ABSTRACT

The present invention relates to novel compositions comprising, (i) at least one of Taxol, Taxotere, bleomycin, carmustine, carboplatin, and doxorubicin; and (ii) MGd, a compound of Formula I. The present invention also relates to and methods of using said compositions to treat Cancer.

### CLAIM OF PRIORITY

The present application claims the benefit of priority from U.S. Provisional Patent Application Serial No. 60/339,649, the contents of which are incorporated herein in their entirety.

### FIELD OF INVENTION

The present invention relates to novel compositions and methods of using these compositions to treat cancer.

### BACKGROUND OF THE INVENTION

Conventional combination chemotherapy regimens have had modest impact on the survival of patients suffering from malignant gliomas, as reported by C. Nieder, A. L. Grosu and M. Molls, “A comparison of treatment results for recurrent malignant gliomas,” Cancer Treatment Reviews, vol. 26, pp. 397-409 (2000). Among the newer chemotherapeutic agents, Taxol has demonstrated significant activity against metastatic non-small cell lung cancer as a single agent and has improved the median survival time of patients (R. S. Herbst, H. Takeuchi and B. A. Teicher “Paclitaxel/carboplatin administration along with antiangiogenic therapy in non-small-cell lung and breast carcinoma models,” Cancer Chemother Pharmacol., vol. 41, pp. 497-504 (1998)).

Texaphyrins have been described as aromatic pentadentate benzannulene compounds containing both 18π- and 22π-electron delocalization pathways, which have the ability to integrate metals within their core to form complexes known as “metallotexaphyrins.” While a variety of metals have been described in forming metallotexaphyrins, the preferred metals have been the lanthanides (and lanthanoids, such as Y³⁺), most notably Gd³⁺ and Lu³⁺. Texaphyrins and metallotexaphyrins have been described, among other things, as chemosensitizers in both cancer and arteriosclerosis treatment, and as photosensitizers in photodynamic therapy of cancer, atherosclerosis, and ophthalmology.

Given the impact of cancer on human life, there is a continuing need to find therapies that will aid in treating cancer and its symptoms.

### SUMMARY OF THE INVENTION

It has been surprisingly found that administering known anti-cancer compounds like Taxol along with texaphyrins improves anticancer efficacy of these drugs.

The present invention thus relates to compositions useful in cancer therapy. Also provided by the present invention are methods of treating a host in need of cancer therapy using the compositions of the present invention.

The present invention provides compositions comprising at least one of a tubulin, stabilizing agent, epothilones, alkylating agent, and thymidylate synthase inhibitor along with a Texaphyrin of Formula I. Another aspect of the present invention provides a method of treating cancer in a patient in need thereof, said treatment comprising administering a therapeutically effective amount of at least one of a tubulin stabilizing agent, epothilones, alkylating agent, and thymidylate synthase inhibitor and a Texaphyrin of Formula I.

### DETAILED DESCRIPTION OF THE INVENTION

The present invention provides composition comprising,

(i) at least one of Taxol, Taxotere, bleomycin, carmustine, carboplatin, and doxorubicin; and

(ii) a compound of Formula I

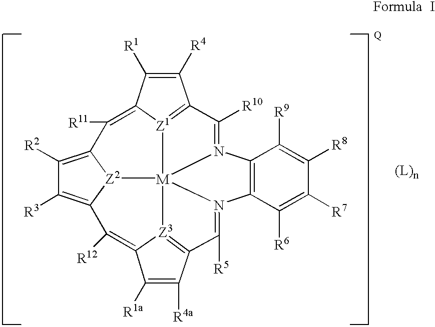

its hydrate, pharmaceutically acceptable salt or prodrug form thereof, wherein:

M represents H or a metal cation;

Q represents an integer of from about −5 to about +5;

L represents a charge balancing species;

n represents an integer of from 0 to +5;

Z¹, Z² and Z³ independently represent N, O, CH or S;

R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO₂R—³¹ , and the moiety X—Y;

R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X—Y;

R⁵, R¹⁰, R¹¹ and R¹² are independently selected from acyl, optionally substituted alkoxy, optionally substituted alkyl, optionally substituted aryl, halo, hydrogen, hydroxy, optionally substituted cycloalkyl, optionally substituted cycloalkenyl, optionally substituted heteroaryl, and optionally substituted heterocyclyl;

X is a covalent bond or a linker;

Y is a catalytic group, a chemotherapeutic agent or a site-directing group;

R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl.

A preferred embodiment provides a composition comprising Taxol or Taxotere and a compound of Formula I:

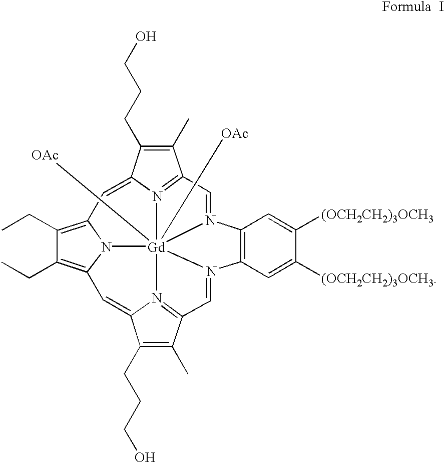

Another preferred embodiment provides a composition comprising one of carmustine and carboplatin and a compound of Formula I:

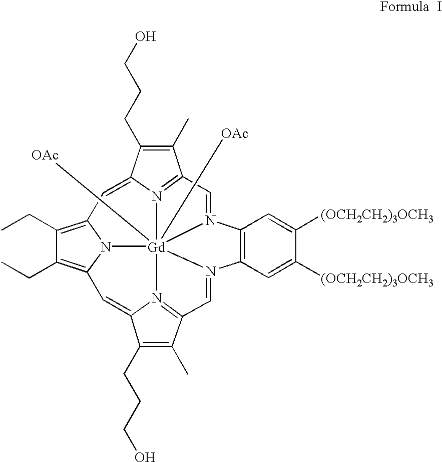

Yet another preferred embodiment provides a composition comprising bloemycin and a compound of Formula I:

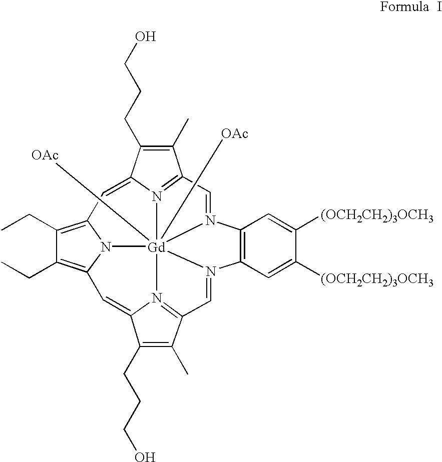

Another preferred embodiment provides a composition comprising doxorubicin and a compound of Formula I:

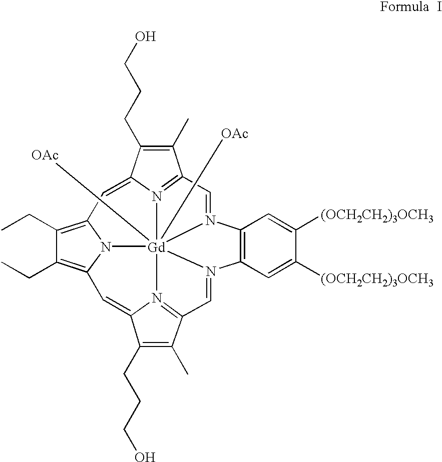

In another aspect of the present invention is provided a method of treating cancer, said method comprising administering to a host in need of such treatment: a compound of Formula I

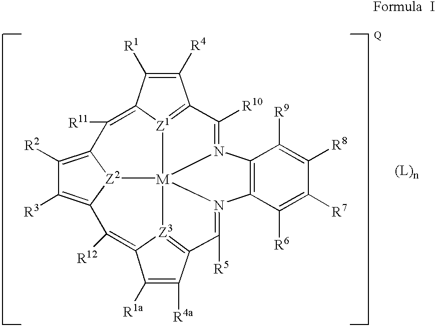

its hydrate, pharmaceutically acceptable salt or prodrug form thereof, wherein:

M represents H or a metal cation;

Q represents an integer of from about −5 to about +5;

L represents a charge balancing species;

n represents an integer of from 0 to +5;

Z¹, Z² and Z³ independently represent N, O, CH or S;

R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO₂—R³¹, and the moiety X—Y;

R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X—Y;

R⁵, R¹⁰, R¹¹ and R¹² are independently selected from acyl, optionally substituted alkoxy, optionally substituted alkyl, optionally substituted aryl, halo, hydrogen, hydroxy, optionally substituted cycloalkyl, optionally substituted cycloalkenyl, optionally substituted heteroaryl, and optionally substituted heterocyclyl;

X is a covalent bond or a linker;

Y is a catalytic group, a chemotherapeutic agent or a site-directing group;

R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl;

in combination with an anticancer agent selected from the group consisting of tubilin stabilizing agents, alkylating agents, thymidylate synthase inhibitors, epidermal growth factors, cyclin dependent kinase inhibitors, DNA cross linking agents(cisplatin, etc.), doxorubicin, bleomycin, spindle poisons(Vepesid) and antimetabolites(gemcitabine).

Another aspect of the present invention provides a method of treating cancer, said method comprising administering to a host in need of such treatment:

(i) a therapeutically effective amount of at least one of Taxol, Taxotere, bleomycin, carmustine, carboplatin, and doxorubicin; and

(ii) a therapeutically effective amount of a compound of Formula I

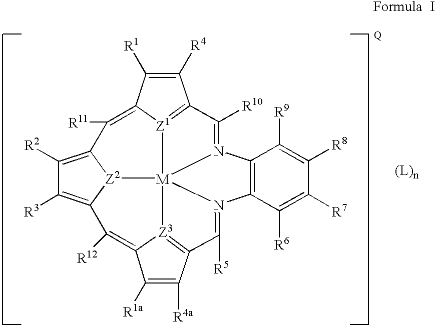

its hydrate, pharmaceutically acceptable salt or prodrug form thereof, wherein:

M represents H or a metal cation;

Q represents an integer of from about −5 to about +5;

L represents a charge balancing species;

n represents an integer of from 0 to +5;

Z¹, Z²and Z³ independently represent N, O, CH or S;

R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO₂—R³¹, and the moiety X—Y;

R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X—Y;

R⁵, R¹⁰, R¹¹ and R¹² are independently selected from acyl, optionally substituted alkoxy, optionally substituted alkyl, optionally substituted aryl, halo, hydrogen, hydroxy, optionally substituted cycloalkyl, optionally substituted cycloalkenyl, optionally substituted heteroaryl, and optionally substituted heterocyclyl;

X is a covalent bond or a linker;

Y is a catalytic group, a chemotherapeutic agent or a site-directing group;

R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl.

A preferred embodiment provides a method of treating cancer said method comprising administering to a host, in need of such treatment, a therapeutically effective amount of Taxol or Taxotere and a therapeutically effective amount of a compound of Formula I:

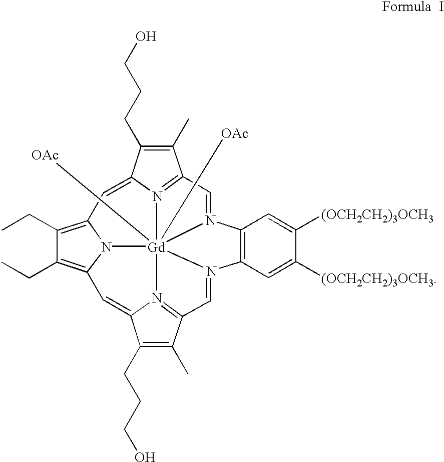

In another preferred embodiment is provided a method of wherein the host is administered, in succession, a therapeutically effective amount of Taxol or Taxotere and a therapeutically effective amount of a compound of Formula I:

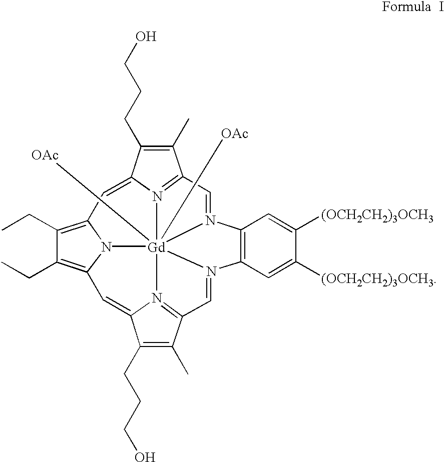

In yet another preferred embodiment is provided a method wherein the host is administered a therapeutically effective amount of Taxol or Taxotere and after about a 2 hour interval a therapeutically effective amount of a compound of Formula I:

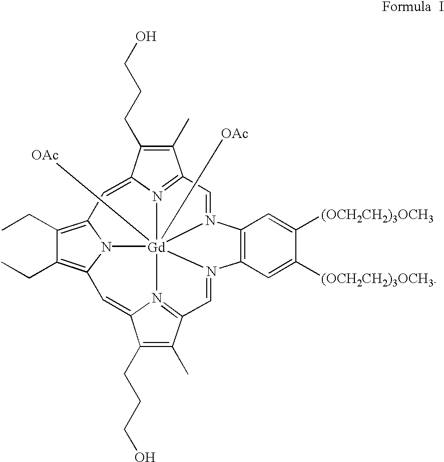

Yet another preferred embodiment provides a method wherein the host is administered a therapeutically effective amount of a compound of Formula I:

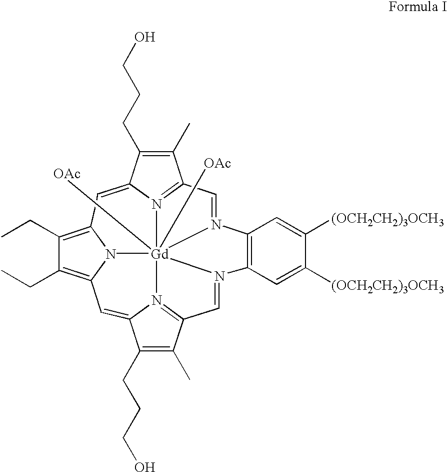

and after a two hour interval a therapeutically effective amount of Taxol or Taxotere.

Yet another preferred embodiment provides a method wherein the host is administered, in succession, a therapeutically effective amount of Taxotere and a therapeutically effective amount of a compound of Formula I:

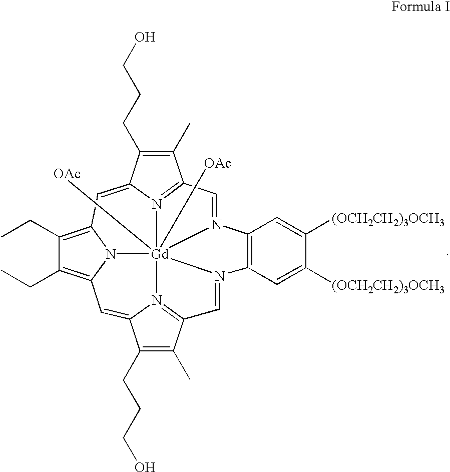

Another aspect of the present invention provides a method of treating cancer, said method comprising administering to a host in need of such treatment a therapeutically effective amount of bleomycin, doxorubicin or carboplatin, and a therapeutically effective amount of a compound of Formula I:

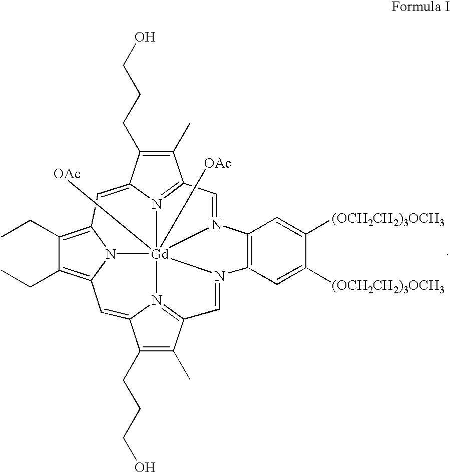

A preferred embodiment provides a method wherein the host is administered, in succession, a therapeutically effective amount of bleomycin, doxorubicin or carboplatin and a therapeutically effective amount of a compound of Formula I:

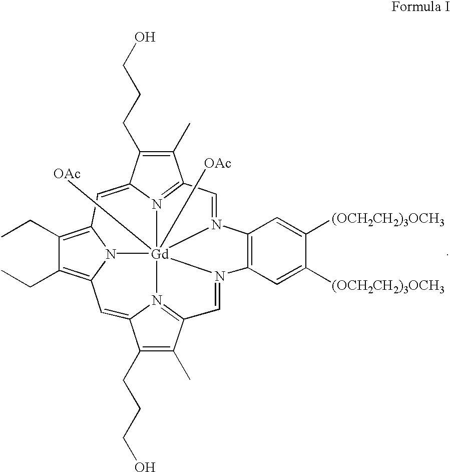

Another preferred embodiment provides a method wherein the host is administered a therapeutically effective amount of bleomycin, doxorubicin or carboplatin and after about a 2 hours interval a therapeutically effective amount of a compound of Formula I:

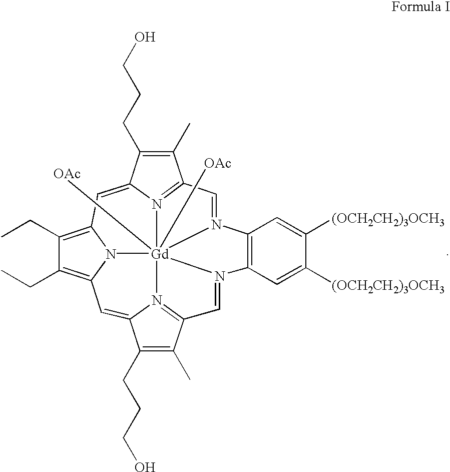

Yet another preferred embodiment provides a method wherein the host is administered a therapeutically effective amount of a compound of Formula I:

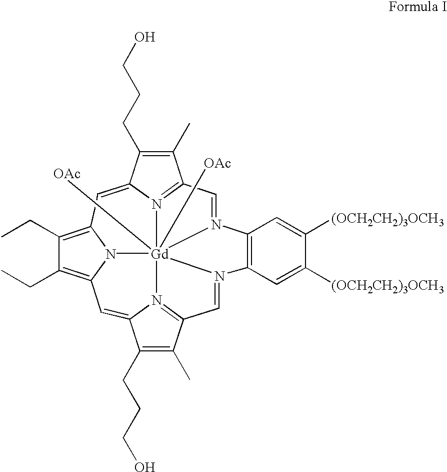

and after a two hour interval a therapeutically effective amount of bleomycin, doxorubicin or carboplatin.

### EXPERIMENTAL

### Examples

Compounds of Formula I, some times also known as Texaphyrins, can be prepared by synthetic procedures outlined in U.S. Pat. No. 5,801,229, the entire contents of are incorporated herein by reference. The following example was prepared using the synthetic procedures mentioned above:

Compound 1: Motexafin Gadolinium (MGd)

The following structure represents Motexafin Gadolinium (MGd), a compound of Formula I.

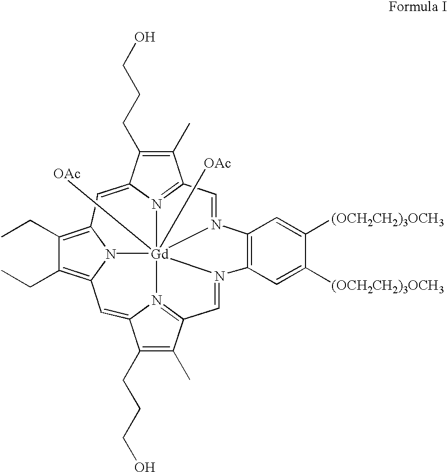

Activity Determination

1. Taxol and MGd

Potential adjuvant effect of combining Taxol chemotherapy with MGd (compound of Formula I) administration was studied in murine lung cancer model as discussed below.

Animals and Tumor Model:

MGd was formulated as a 2 mM solution in 5% aqueous mannitol, pH adjusted to 5.5 with acetic acid. Taxol will be obtained from Sigma Chemical Company (St. Louis, Mo.) and was dissolved in a mixture of 50% Cremophor EL and 50% anhydrous ethanol and then further diluted with saline to give a final concentration of 0.03-0.06 mg/ml. Taxol was filtered with a 0.2 micron in-line filter before use.

Animals

Female C57BL mice weighing 18-20 grams, 9-11 weeks in age were used.

Tumor Model

The murine Lewis lung carcinoma cell line was obtained from American Type Culture Collection (ATCC, Manassas, Va.) (ATTC designation: LLC1, H-2b). The cells wee cultured as mono layers in 75-cm² tissue culture flasks containing Dulbecco's modified eagle's medium with 10% fetal bovine serum, and maintained at 37° C. in a humidified atmosphere containing 5% CO₂ in air. The cell line had been tested and was negative for ectromelia virus (mouse pox). Cells were utilized prior to the tenth passage. The doubling time was 21 hours. A sub cultivation ratio of 1:4 to 1:6 is recommended with the medium being renewed 2 to 3 times per week.

Subcutaneous Tumor Implantation:

The right hind leg of the mouse was shaved and depilated with Nair the day prior to tumor inoculation. The tumor cells (0.5-1×10⁶ in media) were injected subcutaneously into the right hind flanks of the recipient mice. The tumor volume, V, was measured with a vernier caliper. The length (l), width (w), and height (h) were also measured. Tumor volume was calculated assuming the conformation of a hemiellipsoid: V=π/6×(l)×(w)×(h). The animals were placed on study according to each of the different dosing regimens outlined below. The progress of each tumor was monitored thrice weekly.

An end-point study to compare the tumor re-growth delay between the test and control animals was performed. The conditions of all animals were monitored and recorded until the tumor reached 500 mm. Animals with tumor measurement below 500 mm were studied for 60 days post initiation of treatment at which time they were euthanized. When tumor measured 500 mm, the animal was euthanized by carbon dioxide inhalation or other SOP-approved method.

Chemotherapeutic Dosing Regimen

When the tumors are well established, drug administration was initiated about 7-10 days after tumor cell implantation. Taxol (24 mg/kg) was administrated by intravenous injection on days 7, 9, 11 and 13.

Study Groups

There were six study groups with 8 animals in each group. A total of 6 groups were utilized with 8 animals in each group for a total of 48 animals in this study. MGd was intravenously injected at a dose 20 umol/kg while Taxol was injected at a dose of 24 mg/kg.

Group #1 Control Group:

Mice in this group were not administered any Taxol or MGd.

Group #2:

MGd (20 umol/kg) was administered on days 7, 9, 11 and 13.

Group #3:

Taxol (24 mg/kg) was administered on days 7, 9, 11 and 13.

Group #4:

MGd and Taxol were consecutively administered on days 7, 9, 11 and 13.

Group #5:

MGd was first administered, followed by Taxol after a 2hr interval on days 7, 9, 11and 13.

Group #6:

Taxol was administered first, followed by MGd after a 2 hr interval on days 7, 9, 11 and 13:

Results

Results of the above experiment are depicted in Figure I. It was found that the animals in:

Group #1 survived for a median of 11.40 days;

Group #2 (which were administered 20 umol/Kg of MGd on days 7, 9, 11 and 13) survived for a median of 12.90 days;

Group #3 (which were administered 24 umol/Kg of Taxol on days 7, 9, 11 and 13) survived for a median of 11.55 days;

Group #4 (which were consecutively administered 20 umol/Kg of MGd and 24 umol/Kg of Taxol on days 7, 9, 11 and 13) survived for a median of 14.49 days;

Group #5 (which were administered 20 umol/Kg of MGd and then after a 2 hour interval 24 umol/Kg of Taxol on days 7, 9, 11 and 13) survived for a median of 15.84 days; and

Group #6 (which were administered 24 umol/Kg of Taxol and then after a 2 hr interval 20 umol/Kg of MGd on days 7, 9, 11 and 13) survived for a median of 17.56 days.

2. BCNU and MGd

Evaluation of enhancement in the therapeutic activity of the chemotherapeutic agent BCNU (carmustine) by its co-administration with motexafin gadolinium (MGd) was done as discussed below.

Animals and Tumor Model

Test Articles

MGd was formulated as a 2 mM solution in 5% aqueous mannitol, pH adjusted to 5.5 with acetic acid. BCNU will be obtained from Sigma Chemical Company (St. Louis, Mo.).

Animals

Female C57BL mice weighing 18-22 grams, 9-11 weeks in age were used.

Tumor Model

The murine Lewis lung carcinoma cell line was obtained from American Type Culture Collection (ATCC, Manassas, Va.) (ATTC designation: LLC 1, H-2b). The cells were cultured as mono-layers in 75-cm² tissue culture flasks containing Dulbecco's modified eagle's medium with 10% fetal bovine serum, and maintained at 37° C. in a humidified atmosphere containing 5% CO₂ in the air. The cell line was negative for ectromelia virus (mouse pox) and cells were utilized prior to the tenth passage. The doubling time was 21 hours. A sub-cultivation ratio of 1:4 to 1:6 was recommended with the medium being renewed 2 to 3 times per week.

Subcutaneous Tumor Implantation:

The right hind leg of the mouse was shaved and depilated with Nair the day prior to tumor inoculation. The tumor cells (0.5-1×10⁶ in media) were injected subcutaneously into the right hind flanks of the recipient mice. The tumor volume, V, was measured with a vernier caliper. The length (l), width (w), and height (h) were measured. Tumor volume was calculated assuming the conformation of a hemiellipsoid: V=π/6×(l)×(w)×(h). The animals were placed on study according to each of the different dosing regimens outlined below. The progress of each tumor was measured thrice weekly.

Due to the therapeutic nature of this experiment, an end-point study to compare the tumor regrowth delay between the test and control animals was performed. The conditions of all animals were monitored and recorded until the tumor reached 500 mm³. Animals were studied on a continual basis if the tumor measured below 500 mm³, for 60 days post initiation of treatment at which time they were euthanized. Once the tumor measured 500 mm³, the animal was euthanized by an AVMA approved method.

Chemotherapeutic Dosing Regimen

Drug administration was commenced 10 days post tumor cell inoculation. BCNU (15 mg/kg) was injected intraperitoneally.

Study Groups

There were six study groups with 8 animals in each group. A total of 6 groups were studied/utilized with 8 animals in each group for a total of 48 animals in this study. MGd was intravenously injected at a dose 20 umol/kg while BCNU was injected at a dose of 15 mg/kg.

Group #1 Control Group:

Mice in this group were not administered any BCNU or MGd.

Group #2:

MGd (20 umol/kg) was administered on days 10, 12 and 14.

Group #3:

BCNU (15 mg/kg) was administered on days 10, 12 and 14.

Group #4:

MGd and BCNU were consecutively administered on days 10, 12 and 14.

Group #5:

MGd was first administered, followed by BCNU after a 2 hour interval on days 10, 12 and 14.

Group #6:

BCNU was administered first, followed by MGd after a 2 hour interval on days 10, 12 and 14.

Results

Results of the above experiment are depicted in Figure II. It was found that the animals in:

Group #1 survived for a median of 11.45 days;

Group #2 (which were administered 20 umol/Kg of MGd on days 10, 12 and 14) survived for a median of 10.04 days;

Group #3 (which were administered 15 mg/Kg of BCNU on days 10, 12 and 14) survived for a median of 10.46 days;

Group #4 (which were consecutively administered 20 umol/Kg of MGd and 15 mg/kg of BCNU on days 10, 12 and 14) survived for a median of 14.22 days;

Group #5 (which were administered 20 umol/Kg of MGd and then after a 2 hour interval 15 mg/kg of BCNU on days 10, 12 and 14) survived for a median of 14.53 days; and

Group #6 (which were administered 15 mg/kg of BCNU and then after a 2 hour interval 20 umol/Kg of MGd on days 10, 12 and 14) survived for a median of 13.20 days.

3. Bloemycin and MGd

Evaluation of enhancement in the therapeutic activity of the chemotherapeutic agent Bloemycin by its co-administration with motexafin gadolinium (MGd) in the Lewis Lung Cancer (LLC) model was done as discussed below.

Test Articles

Gd-Tex was formulated as a 2 mM solution in 5% aqueous mannitol, pH adjusted to 5.5 with acetic acid. Bleomycin was obtained from Sigma Chemical Company (St. Louis, Mo.).

Animals

Female C57BL mice weighing 18-22 grams, 9-11 weeks in age were obtained from Charles River Laboratories (Hollister, Calif.).

Tumor Model

The murine Lewis lung carcinoma cell line was obtained from American Type Culture Collection (ATCC, Manassas, Va.) (ATTC designation: LLC1, H-2b). The cells were cultured as monolayers in 75-cm² tissue culture flasks containing Dulbecco's modified eagle's medium with 10% fetal bovine serum, and maintained at 37° C. in a humidified atmosphere containing 5% CO₂ in air. The cell line was negative for ectromelia virus (mouse pox) and cells were utilized prior to the tenth passage. The doubling time is 21 hours. A sub cultivation ratio of 1:4 to 1:6 is recommended with the medium being renewed 2 to 3 times per week.

Subcutaneous Tumor Implantation:

The right hind leg of the mouse was shaved and depiled with Nair the day prior to tumor inoculation. The tumor cells (0.5-1×10⁶ in media) were injected subcutaneously into the right hind flanks of the recipient mice. The tumor volume, V, was measured with a vernier caliper. The length (l), width (w), and height (h) will be measured. Tumor volume was calculated assuming the conformation of a hemiellipsoid: V=π/6×(l)×(w)×(h). The animals were placed on study according to each of the different dosing regimens outlined below. The progress of each tumor was measured thrice weekly.

Due to the therapeutic nature of this experiment, an end-point study to compare the tumor regrowth delay between the test and control animals was performed. The conditions of all animals were monitored and recorded until the tumor reaches 500 mm³. Animals were studied on a continual basis if the tumor measured below 500 mm³, for 60 days post initiation of treatment at which time they were euthanized. Once the tumor measured 500 mm³, the animal was euthanized by an AVMA approved method.

Chemotherapeutic Dosing Regimen

Drug administration was commenced 8 days post tumor cell inoculation. Bleomycin (10 units/kg) was injected intravenously.

Study Groups

There were 6 study groups and each group contained 8 animals for a total of 48 animals. MGd was intravenously injected at a dose 20 umol/kg while bleomycin was injected at a dose of 10 Units/kg. The dosing regimens were once a week for three weeks.

Group #1 Control Group:

Mice in this group were not administered any Bleomycin or MGd.

Group #2:

MGd (20 umol/kg) was given weekly for three weeks after the tumor was established, usually 7-10 days after the tumor was planted.

Group #3:

Bleomycin (10 mg/kg) was given weekly for three weeks after the tumor was established, usually 7-10 days after the tumor was planted.

Group #4:

MGd and Bleomycin were consecutively administered weekly for three weeks after the tumor was established, usually 7-10 days after the tumor was planted.

Group #5:

MGd was first administered, followed by Bleomycin after a 2 hour interval weekly for three weeks after the tumor was established, usually 7-10 days after the tumor was planted.

Group #6:

Bleomycin was administered first, followed by MGd after a 2 hour interval, weekly for three weeks after the tumor was established, usually 7-10 days after the tumor was planted.

Results

Results of the above experiment are depicted in FIG. 3. It was found that the animals in:

Group #1 survived for a median of 12.11 days;

Group #2 (which were administered 20 umol/kg of MGd thrice at three day intervals after the tumor was established, usually 7-10 days after the tumor was planted) survived for a median of 13.07 days;

Group #3 (which were administered 10 units/kg of Bleomycin thrice at three day intervals after the tumor was established, usually 7-10 days after the tumor was planted) survived for a median of 14.73 days;

Group #4 (which were consecutively administered 10 units/kg of Bleomycin and 20 umol/kg of MGd thrice at three day intervals after the tumor was established, usually 7-10 days after the tumor was planted) survived for a median of 17.52 days;

Group #5 (which were administered 20 umol/kg of MGd and then after an interval of 2 hours 10 units/Kg of Bleomycin thrice at three day intervals after the tumor was established, usually 7-10 days after the tumor was planted) survived for a median of 19.28 days; and

Group #6 (which were administered 10 units/kg of Bleomycin and then after an interval of 2 hours 20 umol/kg of MGd thrice at three day intervals after the tumor was established, usually 7-10 days after the tumor was planted) survived for a median of 22.22 days.

4. Carboplatin and MGd

Studies with murine Lewis lung cancer in C57 mice, similar to the above examples (co-administration of MGd with Taxol, BCNU and Bleomycin), for co-administration of Carboplatin with MGd yielded results indicating synergistic effect due to co-administration of a MGd with a drug known for its anti-cancer activity.

Results

Results of the above experiment are depicted in FIG. 4. It was found that the animals in:

Group #1 survived for a median of 12.14 days;

Group #2 (which were administered 20 1mol/Kg of MGd on day 7, post tumor inoculation) survived for a median of 12.42 days;

Group #3 (which were consecutively administered 20 μmol/Kg of MGd and 50 mg/kg of carboplatin on day 7, post tumor inoculation) survived for a median of 10.62 days;

Group #4 (which were administered 20 ,mol/Kg of MGd and then after an interval of 2 hours 50 mg/kg carboplatin on day 7, post tumor inoculation) survived for a median of 14.29 days;

Group #5 (which were administered 20 μmol/Kg of MGd and then after an interval of 24 hours 50 mg/kg carboplatin on day 7, post tumor inoculation) survived for a median of 13.46 days;

Group #6 (which were administered 50 mg of carboplatin and then after an interval of 2 hours 20 μmol/Kg of MGd on day 7, post tumor inoculation) survived for a median of 15.56 days; and

Group #7 (which were administered 50 mg of carboplatin and then after an interval of 24 hours 20 μmol/Kg of MGd on day 7, post tumor inoculation) survived for a median of 18.20 days.

The above data indicates that Texaphyrins, in particular MGd, may be administered in combination with other anti-cancer and cytotoxic agents and treatments useful in the treatment of cancer or other proliferative diseases. The present invention indicates that in particular the synergistic effect is more pronounced when the anti-cancer and cytotoxic drugs (illustrative examples are Taxol, Taxotere, bleomycin, carmustine, doxorubicin, and carboplatin) and MGd are administered at an interval of about 2 hours.

Illustrative examples of classes of anti-cancer and cytotoxic agents are alkylating agents (such as nitrogen mustards, alkyl sulfones, nitrosoureas, ethylenimines, and triazines), antimetabolites (such as folate antagonists, purine analogues, and pyrimidine analogues), antibiotics (such as anthracyclines, bleomycins, mitomycin, dactinomycin and plicamycin); enzymes (such as L-asparaginase); famesyl-protein transferase inhibitors; hormonal agents (such as glucocorticoids, estorgens/antiestrogens, androgens/antiandrogens, progestins, and luteinizing hormone-releasing antagonists, octreotide acetate; microtubule-disruptor agents (such as ecteinascidins or their analogs and derivatives); microtubule-stabilizing agents (such as paclitaxel (Taxol®), docetaxel (Taxotere®), and epothilones A-F and/or their analogs or derivatives); plant derived products (such as vinca alkaloids, epipodophyllotoxins, taxanes); topoisomerase inhibitors; prenyl-protein transferase inhibitors; and miscellaneous agents such as hydroxyurea, procarbazine, mitotane, hexamethylmelamine, platinum coordination complexes such as cisplatin and carboplatin, and other agents used as anti-cancer and cytotoxic agents such as biological response modifiers, growth factors, immune modulators, and monoclonal antibodies.

Structures of some of the compounds used in the present invention are as given below:

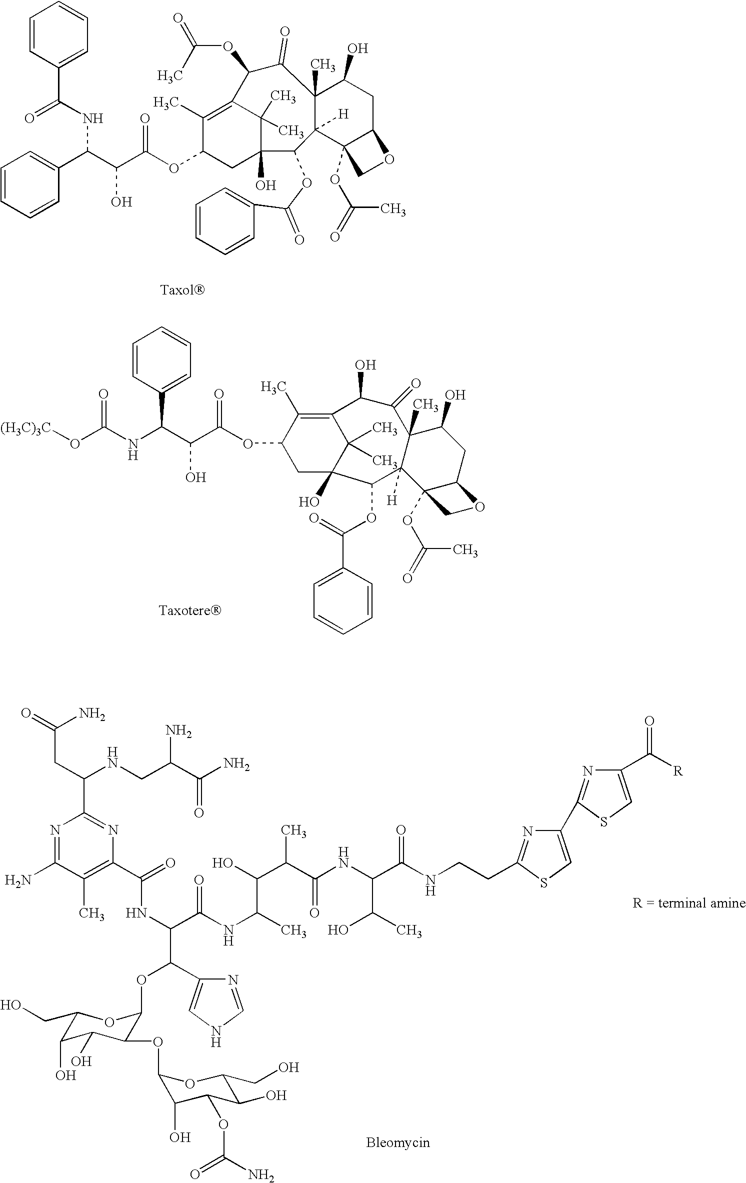

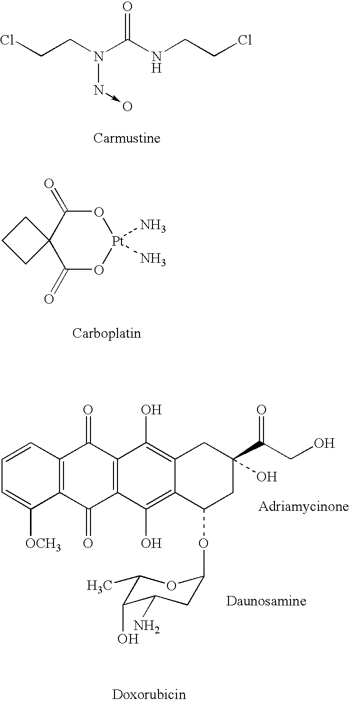

Representative examples of these classes of anti-cancer and cytotoxic agents are mechlorethamine hydrochloride, cyclophosphamide, chlorambucil, melphalan, ifosfamide, busulfan, carmustine, lomustine, semustine, streptozocin, thiotepa, dacarbazine, methotrexate, thioguanine, mercaptopurine, fludarabine, pentastatin, cladribin, cytarabine, fluorouracil, doxorubicin hydrochloride, daunorubicin, idarubicin, bloemycin sulfate, mitomycin C, actinomycin D, safracins, saframycins, quinocarcins, discodermolides, vincristine, vinblastine, vinorelbine tartarate, etoposide, teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, gemcitabine hydrochloride, altretamine, and topoteca and any analogs or derivatives thereof.

Additional examples of anticancer and other cytotoxic agents can be found in German Patent No. 4138042.8; PCT Patent Applications WO 97/19086; WO 98/22461; WO 98/25929; WO 98/38192; WO 99/01124; WO 99/02224; WO 99/02514; WO 99/03848; WO 99/07692; WO 99/27890; WO 99/28324; WO 99/43653; WO 99/54330; WO 99/54318; WO 99/54319; WO 99/65913; WO 99/67252; WO 99/67253; and WO 00/00485; cyclin dependent kinase inhibitors as found in WO 99/24416; and prenyl-protein transferase inhibitors as found in WO 97/30992 and WO 98/54966.

The above therapeutic agents, when employed in combination with the compounds of the present invention, may be used in those amounts indicated in the Physician's Desk reference (PDR) or as otherwise determined by one of ordinary skill in the art.

Definitions

As used in the present specification, the following words and phrases are generally intended to have the meanings as set forth below, except to the extent that the context in which they are used indicates otherwise.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl,” as defined below. It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, the substituents described for RI to R¹² should be generally understood as having a maximum molecular weight of about 1,000 daltons, and more typically, up to about 500 daltons (except in those instances where macromolecular substituents are clearly intended, e.g., polypeptides, polyethylene glycols, DNA, RNA and the like).

The term “acyl” refers to the groups —C(O)—H, —C(O)-(optionally substituted alkyl), —C(O)-(optionally substituted cycloalkyl), —C(O)-(optionally substituted alkenyl), —C(O)-(optionally substituted cycloalkenyl), —C(O)-(optionally substituted aryl), —C(O)-(optionally substituted heteroaryl) and —C(O)-(optionally substituted heterocyclyl).

The term “acyloxy” refers to the moiety —O-acyl, including, for example, —O—C(O)-alkyl.

The term “alkoxy” refers to the groups —O-alkyl, —O-alkenyl, —O-cycloalkyl, —O-cycloalkenyl, and —O-alkynyl. Preferred alkoxy groups are —O-alkyl and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to the groups —O-(substituted alkyl), —O-(substituted alkenyl), —O-(substituted cycloalkyl), —O-(substituted cycloalkenyl), —O-(substituted alkynyl) and —O-(optionally substituted alkylene)-alkoxy. One preferred substituted alkoxy group is “polyalkoxy” or —O-(substituted alkylene)-alkoxy, and includes groups such as —OCH₂CH₂OCH₃, and (or PEG) groups such as —O(CH₂CH₂O)ₓCH₃, where x is an integer of about 2-20, preferably about 2-10, and more preferably about 2-5. Another preferred substituted alkoxy group is —O-(substituted alkyl), and includes groups such as —OCH₂(CH₂)_{y}OH, where y is an integer of about 1-10, preferably about 1-4.

The term “alkoxyalkylene” refers to the groups: -alkylene-O-alkyl,-alkylene-O-(substituted alkyl), -(substituted alkylene)-O-alkyl and -(substituted alkylene)-O-(substituted alkyl). A preferred alkoxyalkylene group is -alkylene-O-alkyl and include, by way of example, methoxymethylene (—CH₂OCH₃), methoxyethylene (—CH₂CH₂OCH₃), n-(iso-propoxy)propylene [—CH₂CH₂CH₂OCH(CH₃)₂] and the like.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having at least 1 and preferably from 1-6 sites of vinyl unsaturation. Preferred alkenyl groups include ethenyl (—CH═CH₂), 2-propen-1-yl (—CH₂CH═CH₂), isopropenyl [—C(CH₃)═CH₂], and the like.

The term “substituted alkenyl” refers to an alkenyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfonyl.

The term “alkenylene” refers to a diradical derived from the above-defined monoradical, alkenyl. This term is exemplified by groups such as ethenylene (—CH═CH—), the propenylene isomers (e.g., —CH₂CH═CH— and —C(CH₃)═CH—) and the like.

The term “substituted alkenylene” refers to a diradical derived from the above-defined monoradical, substituted alkenyl.

The term “alkyl” refers to a monoradical branched or unbranched saturated hydrocarbon chain preferably having from about 1 to 20 carbon atoms, more preferably about 1 to 10 carbon atoms, and even more preferably about 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, phosphono, sulfanyl, sulfinyl, and sulfonyl. One of the preferred optional substituents for alkyl is hydroxy, exemplified by hydroxyalkyl groups, such as 2-hydroxyethyl, 3-hydroxypropyl, 3-hydroxybutyl, 4-hydroxybutyl, and the like; dihydroxyalkyl groups (glycols), such as 2,3-dihydroxypropyl, 3,4-dihydroxybutyl, 2,4-dihydroxybutyl, and the like; and those compounds known as polyethylene glycols, polypropylene glycols and polybutylene glycols, and the like.

The term “alkylene” refers to a diradical derived from the above-defined monoradical, alkyl. This term is exemplified by groups such as methylene (—CH₂—), ethylene (—CH₂CH₂—), the propylene isomers [e.g., —CH₂CH₂CH₂—and —CH(CH₃)CH₂—] and the like.

The term “substituted alkylene” refers to a diradical derived from the above-defined monoradical, substituted alkyl. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethylene (CH(OCH₂CH₃)CH₂), 3-oxapentylene (—CH₂CH₂O—CH₂CH₂—), N-methyl-3-azapentylene (—CH₂CH₂N(CH₃)CH₂CH₂—), 3,6,9-trioxaundecylene (2-ethoxy-ethoxy)ethylene (—CH₂CH₂O—CH₂CH₂—OCH₂CH₂—OCH₂CH₂—), and the like.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl (or propynyl, —C≡CCH₃), and the like.

The term “substituted alkynyl” refers to an alkynyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony.

The term “alkynylene” refers to a diradical derived from the above-defined monoradical, alkynyl. Preferred alkynylene groups include ethynylene (—C≡C—), propargylene (—CH₂—C≡C—) and the like.

The term “substituted alkynylene” refers to a diradical derived from the above-defined monoradical, substituted alkynyl.

The term “amino” refers to the group —NH₂.

The term “substituted amino” refers to the group —NHR or —NRR where each R is independently selected from the group: acyl, optionally substituted alkenyl, optionally substituted alkyl, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, and optionally substituted heterocyclyl. Preferred amino substituents include optionally substituted alkyl, aryl, optionally substituted alkoxycarbonyl (also referred to as a “carbamate”), optionally substituted aminocarbonyl (also referred to as a urea) and heteroaryl.

The term “apical ligand” refers to an anion that binds to the core metal of the metallotexaphyrin, e.g., with de-localized electrostatic or weak coordinate-covalent bonds. The number of apical ligands (n) is defined as an integer of 0-5. It should be noted that the apical ligands act to neutralize the charge on the metallotexaphyrin. Thus, typically n is 1 when M is a divalent cation, and n is 2 when M is a trivalent cation (because the core itself neutralizes one unit charge). However, if any of R¹, R^{1′}, R²R³, R⁴, R^{4′}, R⁵, R⁶, R⁷, R⁸, R⁹, R¹⁰, R¹¹ and R¹² is capable of forming an acid addition salt, for example a carboxylate or a phosphate, then n will decrease appropriately. It is also possible that the apical ligands could have two functionalities capable of forming an anion, for example a dicarboxylic acid, and such ligands are intended to be within the scope of the invention. In general, any molecule containing a carboxylic acid or phosphate may be used as an apical ligand, for example biomolecules, including lipoproteins, estradiol and amino acids, carboxylates of sugar derivatives, such as gluconic acid or glucoronic acid, cholesterol derivatives such as cholic acid and deoxycholic acid, PEG acids, organophosphates, such as methylphosphonic acid and phenylphosphonic acid, and phosphoric acid or other inorganic acids, and the like, or sulfonic acid derivatives such as methanesulfonic acid, ethanesulfonic acid, or “carboxylic acid derivatives”, which term refers to compounds of the formula R—CO₂H, in which R is optionally substituted alkyl, optionally substituted alkenyl, optionally substituted alkynyl, or optionally substituted aryl, as defined above. Preferred are gluconic and glucuronic acid, and those carboxylic acid derivatives where R is optionally substituted alkyl, for example acids of 1-20 carbon atoms, such as formic acid, acetic acid, propionic acid, butyric acid, pentanoic acid, 3,6,9-trioxodecanoic acid, 3,6-dioxoheptanoic acid, methylvaleric acid, glycolic acid, pyruvic acid, oxalic acid, malic acid, malonic acid, succinic acid, maleic acid, fumaric acid, tartaric acid, citric acid, and the like. Also preferred are those carboxylic acid derivatives where R is aryl, in particular where R is optionally substituted phenyl, for example benzoic acid, salicylic acid, 3-fluorobenzoic acid, 4-aminobenzoic acid, cinnamic acid, mandelic acid, p-toluene-sulfonic acid, and the like.

The term “aromatic” refers to a cyclic or polycyclic moiety having a conjugated unsaturated (4n+2)π electron system (where n is a positive integer), sometimes referred to as a delocalized π electron system.

The term “aryl” refers to an aromatic cyclic hydrocarbon group of from 6 to 20 carbon atoms having a single ring (e.g., phenyl) or multiple condensed (fused) rings (e.g., naphthyl or anthryl). Preferred aryls include phenyl, naphthyl and the like.

The term “substituted aryl” refers to an aryl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony. (except as otherwise constrained by the definition for the aryl substituent).

The term “aryloxy” refers to the group —O-aryl.

The term “substituted aryloxy” refers to the group —O-(substituted aryl).

The term “arylalkyl” refers to the moiety “-alkylene-aryl” each having the meaning as defined herein. Such arylalkyl groups are exemplified by benzyl, phenethyl, 3-naphthylpropyl and the like. Arylalkyl moieties also fall within the definition of optionally substituted alkyl, e.g., as a 2-phenyl-n-pentyl moiety.

The term “substituted arylalkyl” refers to the moiety “-(optionally substituted alkylene)-(optionally substituted aryl)”, each having the meaning as defined herein, where at least one of the aryl or alkylene groups is substituted, e.g., 4-(N-methyl-pyrrolyl)pentylene.

The term “carbonyl” refers to the di-radical “—C(═O)—”, which is also written as “—C(O)—”.

The term “(optionally substituted alkoxy)carbonyl” refers to the groups: —C(O)O-(optionally substituted alkyl), —C(O)O-(optionally substituted cycloalkyl), —C(O)O-(optionally substituted alkenyl), and —C(O)O-(optionally substituted alkynyl). These moieties are also referred to as esters.

The term “(optionally substituted amino)carbonyl” refers to the group —C(O)-(optionally substituted amino). This moiety is also referred to as a primary, secondary or tertiary carboxamide.

The term “(optionally substituted alkyl)carbonyloxy” refers to the group —O—C(O)-(optionally substituted alkyl). This moiety is also referred to as a “carbonate.” The term “(optionally substituted amino)carbonyloxy” refers to the group —O—C(O)-(optionally substituted amino). This moiety is also referred to as a “carbamate.”

The term “carboxy” or “carboxyl” refers to the moiety “—C(O)OH”, which is also illustrated as “—COOH”.

The term “compound of Formula I” is intended to encompass the metallotexaphyrins of the invention as disclosed, coordination complexes of the compounds of Formula I, and/or the pharmaceutically acceptable salts of such compounds. In addition, the compounds of this invention include the individual stereochemical isomers and mixtures thereof, arising from the selection of substituent groups.

The term “cycloalkyl” refers to non-aromatic cyclic hydrocarbon groups having about 3 to 40 (preferably about 4 to 15) carbon atoms having a single ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to a cycloalkyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, optionally substituted azo, phosphono, sulfanyl, sulfinyl, and sulfony (except as otherwise constrained by the definition for the cycloalkyl substituent).

The term “cycloalkylene” refers to a diradical derived from the above-defined monoradical, cycloalkyl, and is exemplified by 1,1-cyclopropylene, 1,2-cyclobutylene, 1,4-cyclohexylene and the like.

The term “substituted cycloalkylene” refers to the diradical derived from substituted cycloalkyl as defined above.

The term “halo” or “halogen” refers to fluoro, chloro, bromo and iodo.

The term “heteroaryl” refers to an aromatic cyclic hydrocarbon group having about 1 to 40 (preferably from about 3 to 15) carbon atoms and about 1 to 10 hetero atoms (preferably about 1 to 4 heteroatoms, selected from nitrogen, sulfur, phosphorus, and/or oxygen) within at least one ring. Such heteroaryl groups can have a single ring (e.g., pyridyl or furyl) or multiple condensed rings (e.g., indolizinyl or benzothienyl). Preferred heteroaryls include pyridyl, pyrrolyl and furyl.

The term “substituted heteroaryl” refers to a heteroaryl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony (except as otherwise constrained by the definition for the heteroaryl substituent).

The term “heteroaryloxy” refers to the group —O-heteroaryl.

The term “heteroarylene” refers to the diradical group derived from heteroaryl (including substituted heteroaryl), as defined above, and is exemplified by the groups 2,6-pyridylene, 2,4-pyridylene, 1,2-quinolinylene, 1 ,8-quinolinylene, 1,4-benzofuranylene, 2,5-pyridylene, 2,5-indolylene and the like.

The terms “heterocycle”, “heterocyclic” and “heterocyclyl” are interchangeable, and refer to a monoradical, saturated or unsaturated, non-aromatic cyclic hydrocarbon group having from about 3 to about 40 (preferably from about 3 to about 15) carbon atoms wherein one to about 10 carbon atoms are independently replaced hetero atoms selected from nitrogen, sulfur, phosphorus, oxygen, and selenium. In a preferred embodiment about 1 to about 4 carbon atoms are replaced by hetero atoms. Such heterocyclic groups can have a single ring or multiple condensed rings. Illustrative examples of a heterocycle are morpholino, piperidinyl, and the like.

The terms “substituted heterocycle”, “substituted heterocyclic” and “substituted heterocyclyl” refer to a heterocyclyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, optionally substituted azo, phosphono, sulfanyl, sulfinyl, and sulfony (except as otherwise constrained by the definition for the heterocyclic substituent).

The term “heterocyclylooxy” refers to the group —O-heterocycle.

The term “heterocyclylene” refers to the diradical group formed from a heterocycle, as defined herein, and is exemplified by the groups 2,6-morpholino, 2,5-morpholino and the like.

The term “linker” as used herein means a covalent connection of a functional group (e.g., a site directing group, a catalytic group or a chemotherapeutic agent) to a metallotexaphyrin or its analogue, and may be, for example, a covalent bond or an alkylene, alkenylene, alkynylene, arylene, ether, PEG moiety, and the like, all of which may be optionally substituted. Examples of reactions to form a covalent link include the reaction between an amine (on either the functional group or the linker precursor) with a carboxylic acid (on the other) to form an amide link. Similar reactions well known in the art are described in standard organic chemistry texts such as J. March, “Advanced Organic Chemistry,” 4^{th} Ed., (Wiley-Interscience (New York), 1992).

Dashed lines in cyclic structures indicate optional unsaturation without violating valency rules. Thus in the following structure

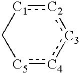

the dashed lines between C₁ and C₂, C₂ and C₃, and C₄ and C₅ respectively indicate that a double bond may or may not exist between all or just a couple of carbon atoms numbered C₁ and C₂, C₂ and C₃, and C₄ and C₅ respectively, as long as the valency rules are not violated.

The term “macrocycle” as used herein refers to a class of polypyrrolic macrocycles that are capable of forming stable complexes with metals by incorporating a metal (as its cation) within a central binding cavity (core) of the macrocycle, and the anions associated with the metal cation are found above and below the core; these anions are known as apical ligands. This class of macrocycles includes porphyrins, the so-called “expanded porphyrins”, and similar structures. Specific examples are porphyrins, porphyrin isomers, porphyrin-like macrocycles, benzophyrins, texaphyrins, alaskaphyrins, sapphyrins, rubyrins, porphycenes, chlorins, benzochlorins, and purpurins.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Pharmaceutically acceptable acid addition salts may be prepared from inorganic and organic acids. The inorganic acids that can be used include hydrochloric acid, hydrobromic acid, sulfuric acid, nitric acid, phosphoric acid, and the like. The organic acids that can be used include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Examples of such pharmaceutically acceptable salts are the iodide, acetate, phenyl acetate, trifluoroacetate, acryl ate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, g-hydroxybutyrate, b-hydroxybutyrate, butyne-1,4-dioate, hexyne-1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like of a compound of formula I.

By “pharmaceutically acceptable” it is also meant that in a formulation containing the compound of Formula l, the carrier, diluent, excipients, and salt must be compatible with the other ingredients of the formulation, and not deleterious to the recipient thereof.

“Texaphyrin” means an aromatic pentadentate macrocyclic expanded porphyrin, also described as an aromatic benzannulene containing both 18π- and 22π-electron delocalization pathways. Texaphyrins and water-soluble texaphyrins, methods of preparation and various uses have been described in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, and 5,714,328; PCT Publications WO 90/10633, 94/29316, 95/10307, 95/21845, 96/09315, 96/40253, 96/38461, 97/26915, 97/35617, 97/46262, and 98/07733; allowed U.S. patent application Ser. Nos. 08/458,347, 08/591,318, and 08/914,272; and pending U.S. patent application Ser. Nos. 08/763,451, 08/903,099, 08/946,435, 08/975,090, 08/975,522, 08/988,336, and 08/975,526; each of which are herein incorporated by reference in their entirety.

Prodrugs are derivatives of the compounds of the invention that have metabolically cleavable groups and become by solvolysis or under physiological conditions the compounds of the invention that are pharmaceutically active in vivo. For example, ester derivatives of compounds of this invention are often active in vivo, but not in vitro. Other derivatives of the compounds of this invention have activity in both their acid and acid derivative forms, but the acid derivative form often offers advantages of solubility, tissue compatibility, or delayed release in the mammalian organism (See, Bundgard, H., “Design of Prodrugs,” pp. 7-9, 21-24, Elsevier, Amsterdam (1985)). Prodrugs include acid derivatives well known to practitioners of the art, such as, for example, esters prepared by reaction of the parent acid with a suitable alcohol, or amides prepared by reaction of the parent acid compound with an amine. Simple aliphatic or aromatic esters derived from acidic groups pendant on the compounds of this invention are preferred prodrugs. In some cases it is desirable to prepare double ester type prodrugs such as (acyloxy)alkyl esters or ((alkoxycarbonyl)oxy)alkyl esters.

The term “therapeutically effective amount” refers to the amount of a compound of Formula I that is sufficient to effect treatment, as defined below, when administered to a mammal in need of such treatment. The therapeutically effective amount will vary depending upon the subject and disease condition being treated, the weight and age of the subject, the severity of the disease condition, the particular compound of Formula I chosen, the dosing regimen to be followed, timing of administration, the manner of administration and the like, all of which can readily be determined by one of ordinary skill in the art.

The term “treatment” or “treating” means any treatment of a disease in a mammal, including:

a) preventing the disease, that is, causing the clinical symptoms of the disease not to develop;

b) inhibiting the disease, that is, arresting the development of clinical symptoms; and/or

c) relieving the disease, that is, causing the regression of clinical symptoms.

It is understood that compounds of Formula I (such as MGd) as well as other known anti-cancer agents are used in their pharmaceutically acceptable forms. It is further understood that compounds of Formula I (such as Example 1) can exist in their respective hydrated form.

1. A composition comprising,
(i) at least one of Taxol, Taxotere, bleomycin, carmustine, carboplatin, and doxorubicin; and
(ii) MGd, a compound of Formula I 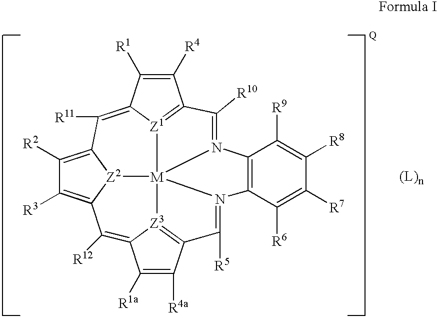
its hydrate, pharmaceutically acceptable salt or prodrug form thereof, wherein:
M represents H or a metal cation;
Q represents an integer of from about −5 to about +5;
L represents a charge balancing species;
n represents an integer of from 0 to +5;
Z¹, Z² and Z³ independently represent N, O, CH or S;
R¹R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO₂—R³¹, and the moiety X—Y;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X—Y;
R⁵, R¹⁰, R¹¹ and R¹² are independently selected from acyl, optionally substituted alkoxy, optionally substituted alkyl, optionally substituted aryl, halo, hydrogen, hydroxy, optionally substituted cycloalkyl, optionally substituted cycloalkenyl, optionally substituted heteroaryl, and optionally substituted heterocyclyl;
X is a covalent bond or a linker;
Y is a catalytic group, a chemotherapeutic agent or a site-directing group;
R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl.

2. A composition of claim 1 comprising Taxol and a compound of Formula I:

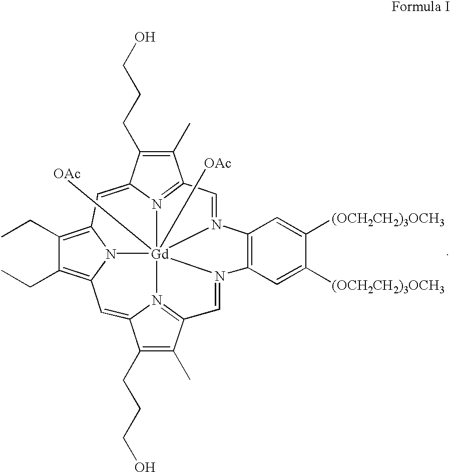

3. A composition of claim 1 comprising Taxotere and a compound of Formula I:

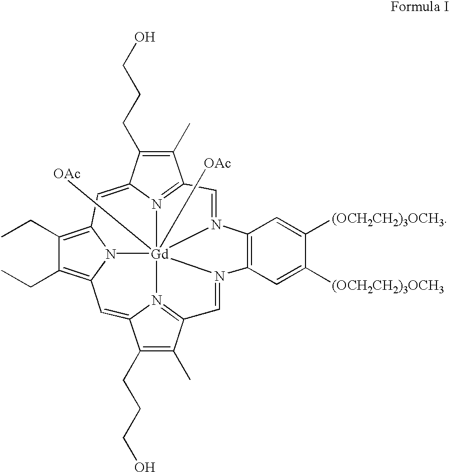

4. A composition of claim 1 comprising bloemycin and a compound of Formula I:

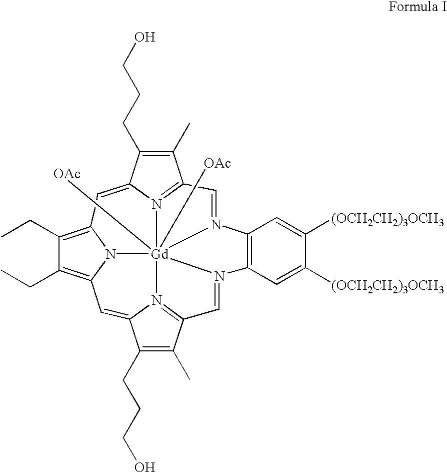

5. A composition of claim 1 comprising doxorubicin and a compound of Formula I:

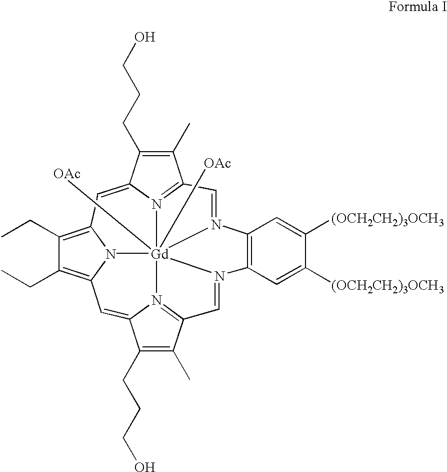

6. A method of treating cancer, said method comprising administering to a host in need of such treatment:
(i) a therapeutically effective amount of at least one of Taxol and Taxotere, bleomycin, and doxorubicin; and
(ii) a therapeutically effective amount of a compound of Formula I 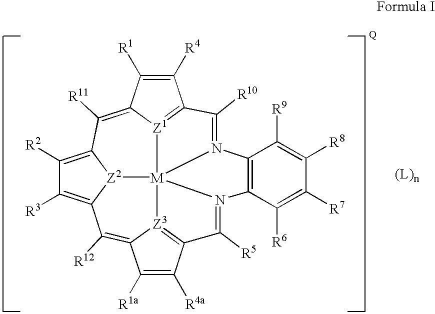
its hydrate, pharmaceutically acceptable salt or prodrug form thereof, wherein:
M represents H or a metal cation;
Q represents an integer of from about −5 to about +5;
L represents a charge balancing species;
n represents an integer of from 0 to +5;
Z¹, Z² and Z³ independently represent N, O, CH or S;
R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO₂—R³¹, and the moiety X—Y;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X—Y;
R⁵, R¹⁰, R¹¹ and R¹² are independently selected from acyl, optionally substituted alkoxy, optionally substituted alkyl, optionally substituted aryl, halo, hydrogen, hydroxy, optionally substituted cycloalkyl, optionally substituted cycloalkenyl, optionally substituted heteroaryl, and optionally substituted heterocyclyl;
X is a covalent bond or a linker;
Y is a catalytic group, a chemotherapeutic agent or a site-directing group;
R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl.

7. A method of treating cancer said method comprising administering to a host, in need of such treatment, a therapeutically effective amount of Taxol and a therapeutically effective amount of a compound of Formula I:

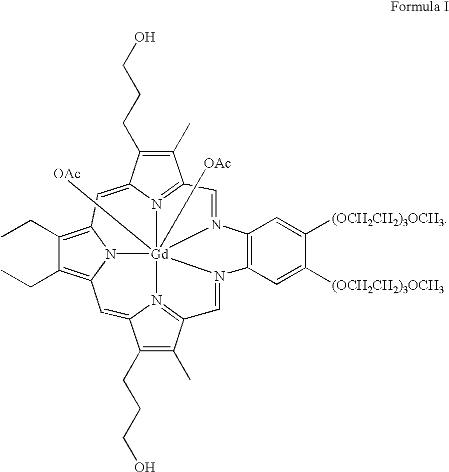

8. A method of claim 7 wherein the host is administered, in succession, a therapeutically effective amount of Taxol and a therapeutically effective amount of a compound of Formula I:

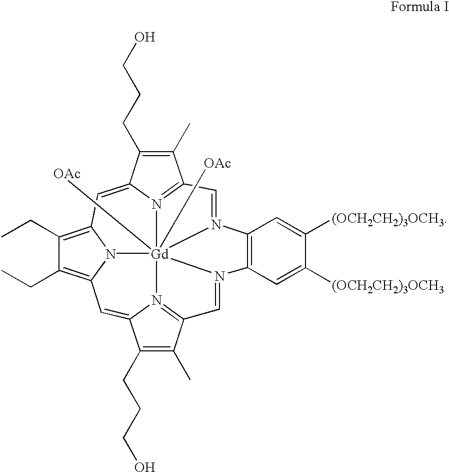

9. A method of claim 7 wherein the host is administered a therapeutically effective amount of Taxol and after about a 2 hours interval a therapeutically effective amount of a compound of Formula I:

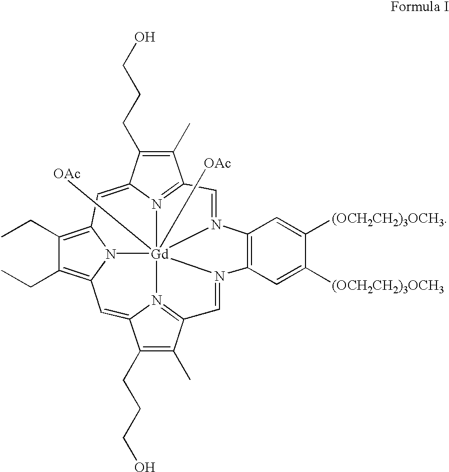

10. A method of claim 7 wherein the host is administered a therapeutically effective amount of a compound of Formula I: 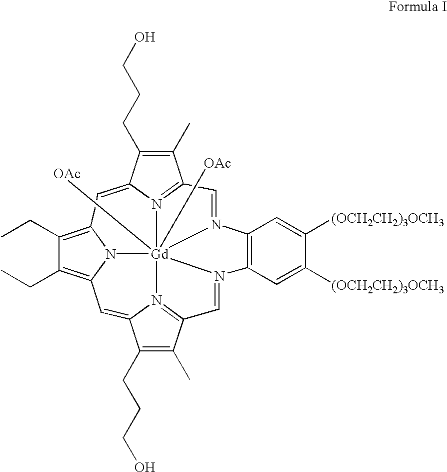
and after a two hour interval a therapeutically effective amount of Taxol.

11. A method of treating cancer, said method comprising administering to a host in need of such treatment a therapeutically effective amount of Taxotere and a therapeutically effective amount of a compound of Formula I:

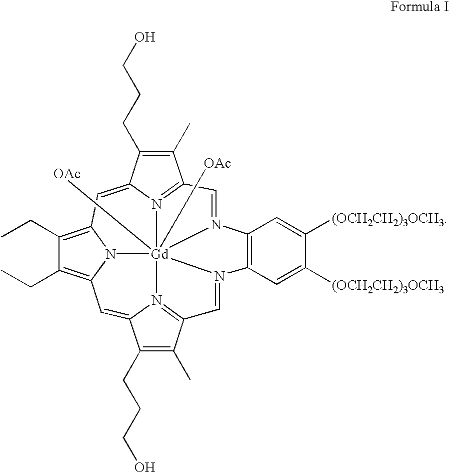

12. A method of claim 11 wherein the host is administered, in succession, a therapeutically effective amount of Taxotere and a therapeutically effective amount of a compound of Formula I:

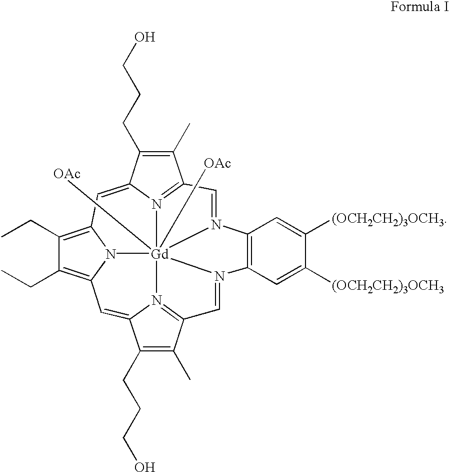

13. A method of claim 11 wherein the host is administered a therapeutically effective amount of Taxotere and after about a 2 hours interval a therapeutically effective amount of a compound of Formula I:

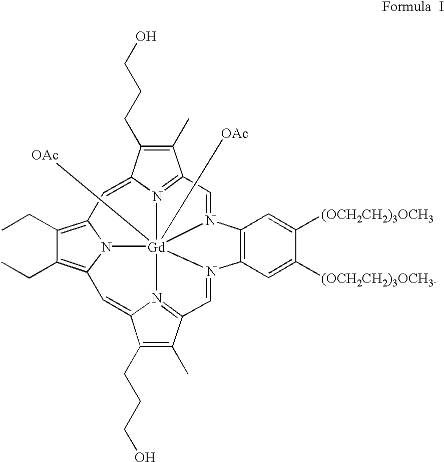

14. A method of claim 11 wherein the host is administered a therapeutically effective amount of a compound of Formula I: 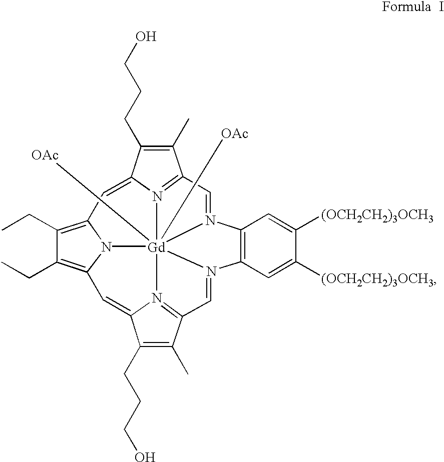
and after a two hour interval a therapeutically effective amount of Taxotere.

15. A method of treating cancer, said method comprising administering to a host in need of such treatment a therapeutically effective amount of bleomycin and a therapeutically effective amount of a compound of Formula I:

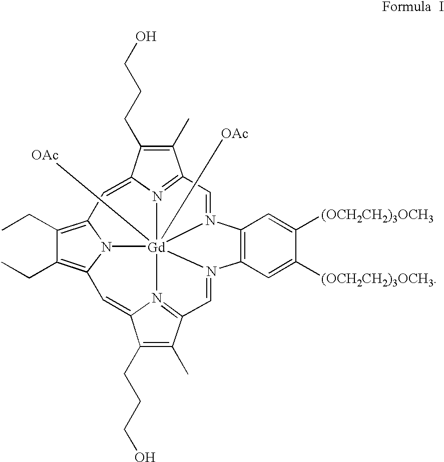

16. A method of claim 15 wherein the host is administered, in succession, a therapeutically effective amount of bleomycin and a therapeutically effective amount of a compound of Formula I:

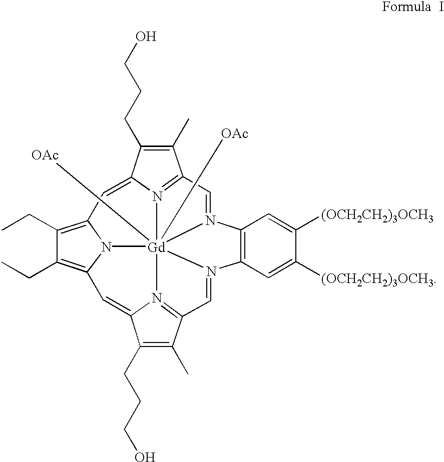

17. A method of claim 15 wherein the host is administered a therapeutically effective amount of bleomycin and after about a 2 hours interval a therapeutically effective amount of a compound of Formula I:

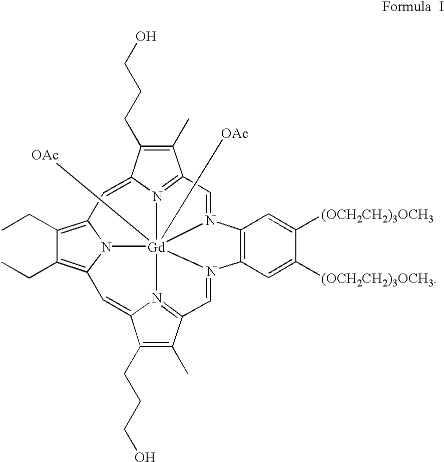

18. A method of claim 15 wherein the host is administered a therapeutically effective amount of a compound of Formula I: 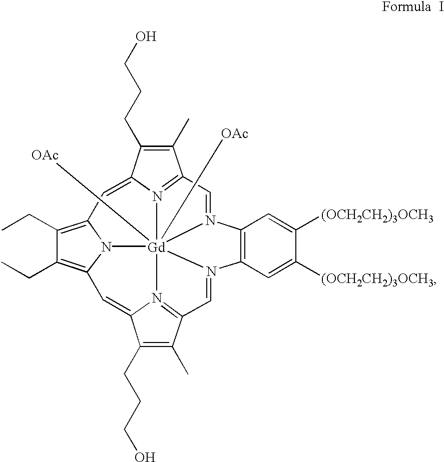
and after a two hour interval a therapeutically effective amount of bleomycin.

19. A method of treating cancer, said method comprising administering to a host in need of such treatment a therapeutically effective amount of doxorubicin and a therapeutically effective amount of a compound of Formula I:

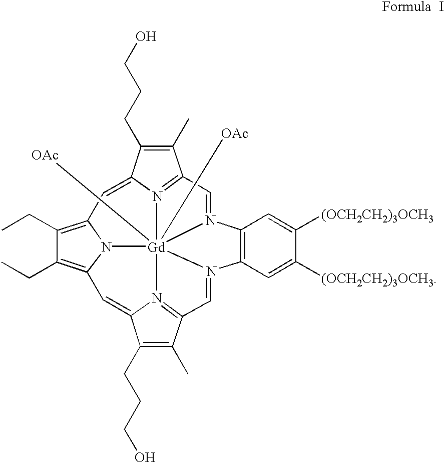

20. A method of claim 19 wherein the host is administered, in succession, a therapeutically effective amount of doxorubicin and a therapeutically effective amount of a compound of Formula I:

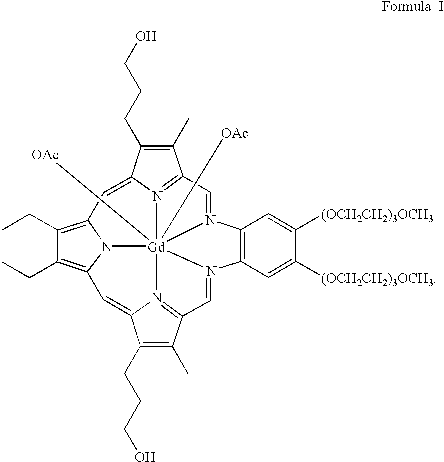

21. A method of claim 19 wherein the host is administered a therapeutically effective amount of doxorubicin and after about a 2 hours interval a therapeutically effective amount of a compound of Formula I:

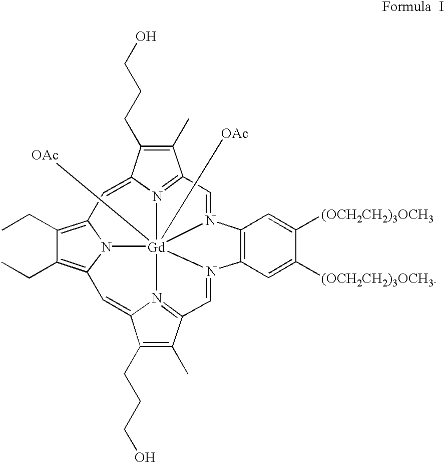

22. A method of claim 19 wherein the host is administered a therapeutically effective amount of a compound of Formula I: 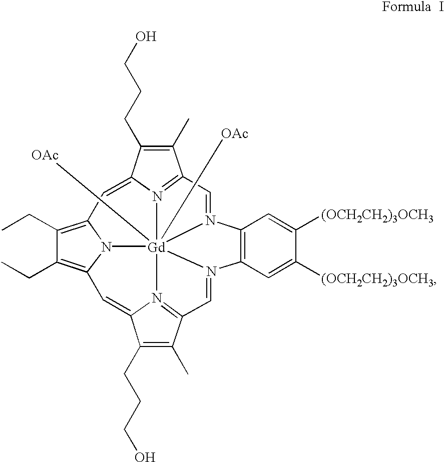
and after a two hour interval a therapeutically effective amount of doxorubicin.
